(19) United States
(12) Patent Application Publication
(10) Pub. No.: US 2018/0015272 A1
(43) Pub. Date: Jan. 18, 2018

(54) MICRONEEDLE WITH POROUS COATING LAYER, MANUFACTURING METHOD THEREOF AND MICRONEEDLE PATCH WITH THE SAME

(71) Applicant: Kyungpook National University Industry-Academic Cooperation Foundation, Daegu (KR)

(72) Inventor: Gyu Man KIM, Daegu (KR)

(73) Assignee: Kyungpook National University Industry-Academic Cooperation Foundation, Daegu (KR)

(21) Appl. No.: 15/630,953

(22) Filed: Jun. 23, 2017

(30) Foreign Application Priority Data
Jul. 14, 2016 (KR) 10-2016-0089333

(51) Int. Cl. A61M 37/00; A61K 9/00; A61K 9/16; A61L 31/14; A61K 9/70

(52) U.S. Cl. CPC A61M 37/0015; A61K 9/16; A61L 31/146; A61K 9/0021; A61M 2037/0053; A61K 9/7023

(57) ABSTRACT

The present invention relates to a microneedle coated with a porous coating layer, a manufacturing method thereof and a microneedle patch with the same, which can secure a channel to deliver medicine in a surface of a needle body and improve an efficiency of collecting and delivering the medicine based on merits of a material.

To this end, the microneedle coated with a porous coating layer includes a needle body hard enough to penetrate an outer skin layer; and a coating layer coated to surround the needle body, wherein the needle body is made of a high-stiffness material including metal or silicon, the coating layer includes a plurality of communicating holes recessed or penetrated to be filled with medicine, and the plurality of communicating holes communicate with one another to form a moving path of the medicine.

### CROSS-REFERENCE TO RELATED APPLICATION

This application claims priority from Korean Patent Application No. 10-2016-0089333, filed on Jul. 14, 2016 in the Korean Intellectual Property Office, the disclosure of which is incorporated herein by reference.

### BACKGROUND OF THE INVENTION

### (a) Field of the Invention

The present invention relates to a microneedle coated with a porous coating layer, a manufacturing method thereof and a microneedle patch with the same, and more particularly to a microneedle coated with a porous coating layer, a manufacturing method thereof and a microneedle patch with the same, which can secure a channel to deliver medicine in a surface of a needle body and improve an efficiency of collecting and delivering the medicine based on merits of a material.

### (b) Description of the Related Art

For transdermal delivery of medicine, a microneedle is generally used. Technology of using the microneedle for the transdermal delivery of the medicine is highly useful in beauty/medical industries, but has a low current efficiency on delivering the medicine to an intradermal layer of skin.

For example, a conventional microneedle may be a hollow-type microneedle that is formed by subminiaturizing an injection needle through micro-machining. In this case, a hollow needle shaped like a tube penetrates an outer skin layer, and then medicine is injected into a hollow channel so that an intradermal layer can absorb the medicine. However, the hollow-type microneedle has problems of requiring precise machining at manufacture, and leading to very difficult machining and cost increase if it is manufactured to have a diameter of 1 mm or less.

For another example, a conventional microneedle may be a solid-type microneedle that is shaped like a pin. In this case, a hole is pricked in an outer skin layer with the needle and then medicine is smeared on the skin so that the medicine can be absorbed in an intradermal layer through the hole formed in the outer skin layer. However, there is a limit to the efficiency on delivering the medicine since the medicine is delivered based on the principle that the medicine is applied to the surface of skin and then absorbed in the skin and the conventional microneedle plays just a role of assisting the absorption of the medicine.

For still another example, a conventional microneedle may be a solid-type microneedle that is shaped like a pin as shown in (a) of FIG. 1, and a microneedle patch 1 may include a pad portion 2 shaped like a plate, a plurality of needle portions 3 protruding from the pad portion 2, and a medicine 4 coated on the surfaces of the pad portion 2 and the needle portion 3.

Like this, the surface of the microneedle patch 1 is coated with the medicine 4, so that the medicine 4 coated on the surface of the needle portion 3 can be absorbed in the intradermal layer after the needle portion 3 penetrates the outer skin layer. This improves an efficiency of delivering the medicine 4, but has shortcomings that the amount of coated medicine 4 is very small. Further, as shown in FIG. 1, when the needle portion 3 penetrates the outer skin layer, the medicine 4 coated on the surface of the needle portion 3 is moved back on the needle portion 3 by the outer skin layer and moves to and stays on the surface of the outer skin layer without getting into the skin. Therefore, the medicine 4 is not properly delivered to the intradermal layer.

Besides, a conventional microneedle may be made of polymer, hydrogel or the like soft material. In addition, a conventional microneedle may be made of a material that is easily melted inside a human body. This conventional microneedle is melted into the intradermal layer after penetrating the outer skin layer. Such a dissolve-type needle is filled with medicine, and thus the medicine is delivered to the intradermal layer as the needle is melted. However, this conventional microneedle has a low efficiency of penetrating the outer skin layer since its material is not hard.

### PRIOR ART DOCUMENTATION

### Patent Documentation

(Patent Documentation) Korean Patent Publication No. 2012-0030055 (titled “MICRONEEDLE DEVICE” and published on Mar. 27, 2012).

### SUMMARY OF THE INVENTION

Accordingly, the present invention is conceived to solve the conventional problems, and an aspect of the present invention is to provide a microneedle coated with a porous coating layer, a manufacturing method thereof and a microneedle patch with the same, which can secure a channel to deliver medicine in a surface of a needle body and improve an efficiency of delivering the medicine based on merits of a material.

In accordance with an embodiment of the present invention, there is provided a microneedle coated with a porous coating layer, which includes a needle body hard enough to penetrate an outer skin layer; and a coating layer coated to surround the needle body, wherein the needle body is made of a high-stiffness material including metal or silicon, the coating layer includes a plurality of communicating holes recessed or penetrated to be filled with medicine, and the plurality of communicating holes communicate with one another to form a moving path of the medicine.

The coating layer may be made of a soft material comprising at least one of biocompatible polymer and biocompatible hydrogel; and water-soluble porogen different in state from the soft material, and the communicating holes may be recessed or penetrated as the porogent is removed from the coating layer.

The biocompatible polymer may comprise at least one of poly lactic-co-glycolic acid (PLGA) and poly lactic acid (PLA), and the biocompatible hydrogel may comprise at least one of poly ethylene glycol (PEG), poly ethylene glycol diacrylate (PEGDA), and poly ethylene glycol methyl ether acrylate (PEGMEA).

The needle body may be shaped like a horn or a column, a tip of which is formed by its material characteristic.

In accordance with an embodiment of the present invention, there is provided a method of manufacturing a microneedle of which a needle body is coated with a coating layer, the method comprising: an emulsion producing operation of dissolving a soft material as a raw material of the coating layer in a solvent and mixing porogen with the dissolved soft material to produce emulsion; an emulsion coating operation of coating the emulsion on a surface of the needle body; an emulsion drying operation of drying the emulsion in the needle body after the emulsion coating operation; and a porogen removing operation of removing the porogen from the emulsion dried in the needle body.

The method further comprising a body manufacturing operation of using a high-stiffness material including metal or silicon to manufacture the needle body,

In accordance with an embodiment of the present invention, there is provided a microneedle patch comprising: a needle path unit comprising a needle pad shaped like a plate, and a needle body protruding from the needle pad and hard enough to penetrate an outer skin layer; and a coating layer coated to surround at least the needle body in the needle patch unit, wherein the needle body is made of a high-stiffness material including metal or silicon, the coating layer comprises a plurality of communicating holes recessed or penetrated to be filled with medicine, and the plurality of communicating holes communicate with one another to form a moving path of the medicine.

### BRIEF DESCRIPTION OF THE DRAWINGS

The above and/or other aspects of the present invention will become apparent and more readily appreciated from the following description of the exemplary embodiments, taken in conjunction with the accompanying drawings, in which:

FIG. 1 is a cross-section view of a conventional microneedle patch and a situation of using the same to deliver medicine;

FIG. 2 is a cross-section view of a microneedle patch according to an embodiment of the present invention;

FIG. 3 is an enlarged photograph of a coating layer to be coated on a needle body in the microneedle patch according to an embodiment of the present invention;

FIG. 4 is an enlarged photograph of a coating layer partially peeled away from the needle body in the microneedle patch according to an embodiment of the present invention;

FIG. 5 is an enlarged cross-section view of showing that medicine is delivered from the microneedle patch according to an embodiment of the present invention; and

FIG. 6 is a graph of showing experimental results between the microneedle according to an embodiment of the present invention and a comparative group with respect to a sample medicine.

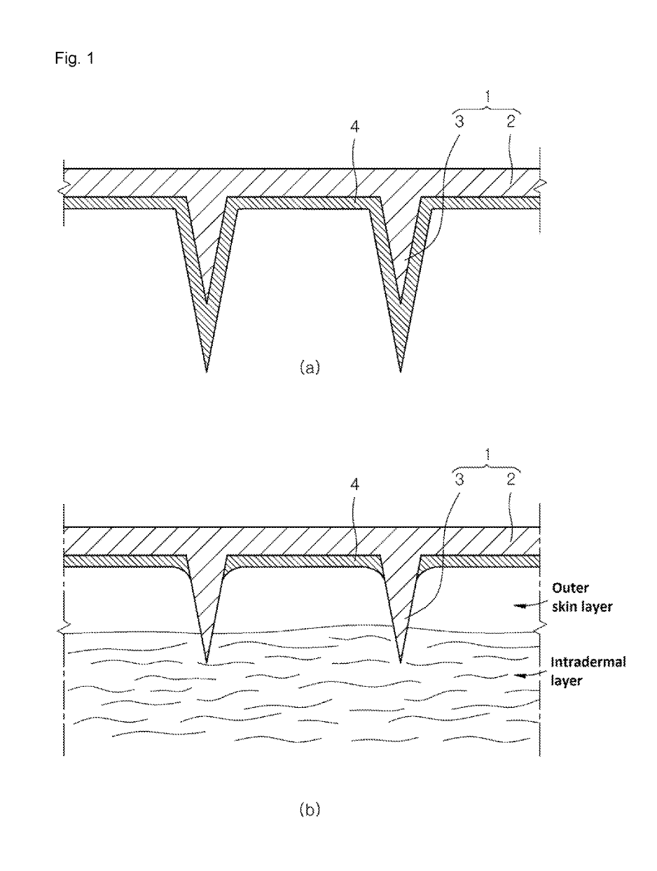

### DETAILED DESCRIPTION OF THE EMBODIMENTS

Hereinafter, embodiments of a microneedle coated with a porous coating layer, a manufacturing method thereof and a microneedle patch with the same according to the present invention will be described with reference to the accompanying drawings. The present invention is not limited or restricted by the following embodiments. Further, detailed descriptions of publicly known functions or structures may be omitted to make the gist of the present invention clear.

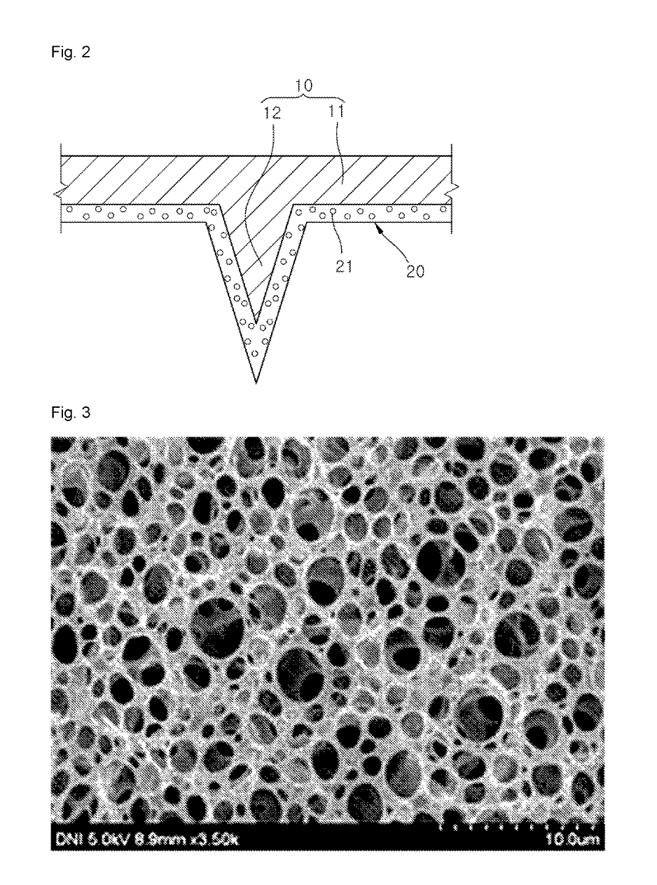

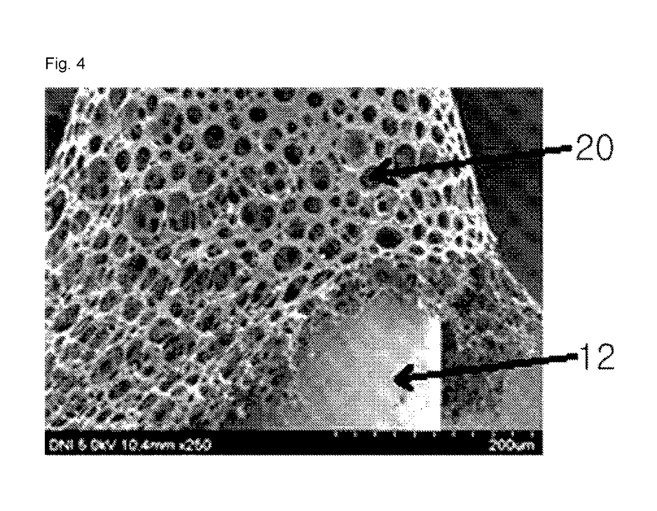

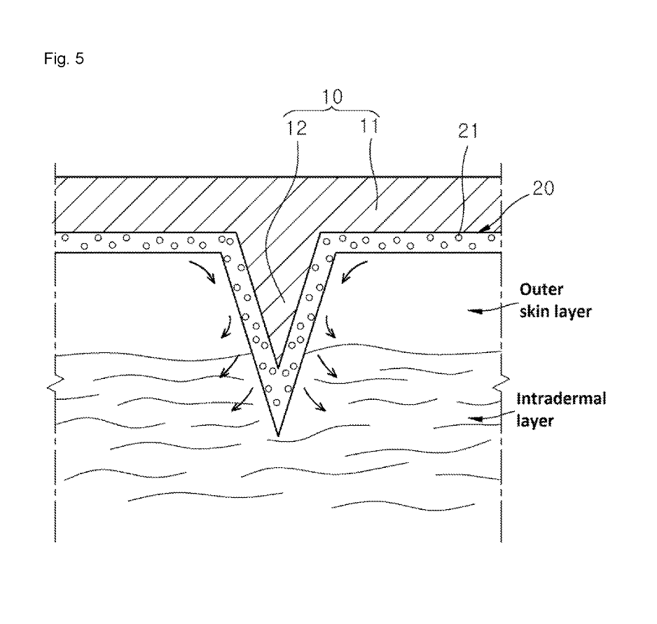

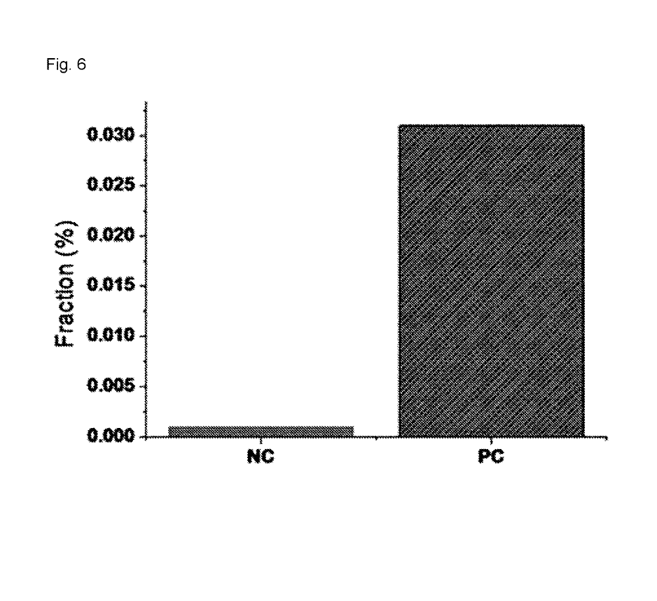

FIG. 2 is a cross-section view of a microneedle patch according to an embodiment of the present invention, FIG. 3 is an enlarged photograph of a coating layer to be coated on a needle body in the microneedle patch according to an embodiment of the present invention, FIG. 4 is an enlarged photograph of a coating layer partially peeled away from the needle body in the microneedle patch according to an embodiment of the present invention, FIG. 5 is an enlarged cross-section view of showing that medicine is delivered from the microneedle patch according to an embodiment of the present invention; and FIG. 6 is a graph of showing experimental results between the microneedle according to an embodiment of the present invention and a comparative group with respect to a sample medicine.

Referring to FIG. 2 to FIG. 6, a microneedle coated with a porous coating layer, a manufacturing method thereof and a microneedle patch with the same according to an embodiment of the present invention will be described

First, the microneedle according to an embodiment of the present invention will be described. The microneedle according to an embodiment of the present invention has a new structure that a porous coating layer 20 is coated on a needle body 12.

The microneedle may include the needle body 12 and the coating layer 20.

The needle body 12 is hard enough to penetrate the outer skin layer. For example, the needle body 12 may be made of a high-stiffness material including stainless steel or the like metal or silicon. Further, the needle body 12 may have tens of micrometers to 500 micrometers in diameter and 100 micrometers to 1 millimeter in length.

Further, the needle body 12 may be shaped like a horn or a column, a tip of which is formed by its material characteristic. In more detail, since the needle body 12 is made of the high-stiffness material including metal or silicon, the needle body 12 can have a horn or column shape, it is easy to form a tip in the needle body 12, and it is convenient to manufacture the needle body 12 through various methods.

For example, as shown in FIG. 2, the needle body 12 may be manufactured to have a cone, an elliptical cone or a poly-pyramid with a tip due to its material characteristic.

For another example, although it is not shown, the needle body 12 may be manufactured to have a cylinder, a cylindroid or a poly-prism with a due to its material characteristic.

The coating layer 20 is coated to surround the needle body 12. The coating layer 20 is formed with a plurality of communicating holes 21 recessed or penetrated to be filled with medicine, thereby achieving the porous coating layer 20. Thus, the medicine is collected in the communicating holes 21.

The plurality of communicating holes 21 communicate with one another to form a moving path of the medicine so that the medicine can be easily filled in and expelled from the coating layer 20. In other words, the plurality of communicating holes 21 are connected to one another and thus form a physical microstructure to collect a large amount of medicine on the surface of the needle body 12 or provide a physical space in which the medicine continuously flows, thereby continuously delivering the medicine to an intradermal layer through the surface of the needle body 12.

The coating layer 20 contains a soft material including at least one of biocompatible polymer and biocompatible hydrogel, and porogen including a material different in state from the soft material. The plurality of communicating holes 21 may be recessed or penetrated as the porogen is removed from the coating layer 20 coated on the needle body 12.

For example, the biocompatible polymer may include at least one of poly lactic-co-glycolic acid (PLGA) and poly lactic acid (PLA). The biocompatible hydrogel may include at least one of poly ethylene glycol (PEG), poly ethylene glycol diacrylate (PEGDA), and poly ethylene glycol methyl ether acrylate (PEGMEA). The porogen may include at least one of water containing phosphate buffer saline (PBS) or the like, water-soluble hydrogel containing gelatin, and salt or the like solid formulation.

With this, the medicine is delivered as follows. The medicine is filled in the communicating holes 21 of the coating layer 20 coated on the needle body 12. In the state that the communicating holes 21 are filled with the medicine, the skin is pricked on the needle body 12. Then, the needle body 12 penetrates the outer skin layer, and the medicine filled in the communicating holes 21 is released to the skin and thus delivered to the intradermal layer.

At this time, the coating layer 20 forms a space between the needle body 12 and the outer skin layer and therefore minimizes loss of the medicine while the needle body 12 is inserted in the outer skin layer. After a lapse of preset time, the needle body 12 is detached from the skin.

Further, the medicine may be refilled in the communicating holes 21 of the coating layer 20 by separating the microneedle from the skin, and the operation of inserting the needle body 12 in the skin may be repeated so as to sufficiently deliver the medicine to the intradermal layer of the skin.

To this end, various methods of filling the communicating holes 21 with the medicine may be used. For example, the coating layer 20 may be immersed in the medicine.

Next, the manufacturing method of the microneedle according to an embodiment of the present invention will be described. As a method of forming the coating layer 20 on the needle body 12, the manufacturing method of the microneedle according to an embodiment of the present invention may include an emulsion producing operation, an emulsion coating operation, an emulsion drying operation, a porogen removing operation and may further include a body manufacturing operation.

The body manufacturing operation is performed prior to the emulsion coating operation. In the body manufacturing operation, the high-stiffness material including metal or silicon is used for manufacturing the needle body 12. Here, the needle body 12 is shaped like a horn or column with a tip. By the body manufacturing operation, the high-stiffness material is processed to have a horn or column shape with the tip through various methods, thereby manufacturing the needle body 12. For example, the needle body 12 manufactured by the body manufacturing operation may be made of a stainless material.

In the emulsion producing operation, a soft material used as a raw material of the coating layer 20 is dissolved in a solvent and mixed with the porogen to produce emulsion.

Here, the soft material may include at least one of the biocompatible polymer and the biocompatible hydrogel.

For example, the biocompatible polymer may include poly lactic-co-glycolic acid (PLGA) and poly lactic acid (PLA). The biocompatible hydrogel may include at least one of poly ethylene glycol (PEG), poly ethylene glycol diacrylate (PEGDA), and poly ethylene glycol methyl ether acrylate (PEGMEA). The porogen may include at least one of water containing phosphate buffer saline (PBS) or the like, water-soluble hydrogel containing gelatin, and salt or the like solid formulation.

Further, the porogen is made of a material different in state from the soft material, and it is thus easy to remove the porogen in the porogen removing operation.

In the emulsion producing operation, it is possible to adjust the porosity of the finally completed coating layer 20 by controlling a ratio of the porogen and the soft material to be mixed in the solvent.

In the emulsion coating operation, the emulsion is coated on the surface of the needle body 12. A method of coating the emulsion may include dip coating, spin coating, spray coating, etc.

By controlling the emulsion coating operation, it is possible to adjust the thickness of the finally completed coating layer 20.

In the emulsion drying operation, the emulsion is dried in the needle body 12 after the emulsion coating operation.

In the porogen removing operation, the porogen is removed from the emulsion dried in the needle body 12. Here, the porogen may be variously removed from the emulsion without limiting the porogen removing method.

Last, the microneedle patch with the microneedle according to an embodiment of the present invention will be described. The microneedle patch with the microneedle according to an embodiment of the present invention may include a needle patch unit 10, and the coating layer 20.

The needle patch unit 10 includes a needle pad 11, and the needle body 12.

The needle pad 11 is shaped like a plate, and the needle body 12 is hard enough to penetrate the outer skin layer and protrudes from the needle pad 11.

The needle pad 11 and the needle body 12 may be made of one material and formed as a single body. Alternatively, the needle pad 11 and the needle body 12 may be made of materials different from each other, and the needle body 12 is integrally coupled to the needle pad 11. In addition, the needle pad 11 and the needle body 12 may be made of materials different from each other, and the needle body 12 may be separated from the needle pad 11 in accordance with external conditions.

The coating layer 20 is coated to surround at least the needle body 12 of the needle patch unit 10. In other words, the coating layer 20 is coated to cover at least the needle body 12 on the surface of the needle pad 11. For example, coating layer 20 may be coated on only the needle body 12. Alternatively, the coating layer 20 may be coated on the needle body 12 and a partial surface of the needle pad 11 coupling with the needle body 12. Alternatively, the coating layer 20 may be coated on the needle body 12 and the entire of one side of the needle pad 11 from which the needle body 12 protrudes.

The coating layer 20 is formed with the plurality of communicating holes 21 recessed or penetrated to be filled with the medicine, thereby achieving the porous coating layer 20. The plurality of communicating holes 21 communicate with one another to form the moving path of the medicine, so that the medicine can be easily filled in and discharged from the coating layer 20.

The material for the coating layer 20 includes the soft material including at least one of the biocompatible polymer and the biocompatible hydrogel, and the water-soluble porogen different in state from the soft material. Further, the communicating holes 21 are recessed or penetrated as the porogen is removed from the coating layer 20 coated on the needle body 12.

For example, the biocompatible polymer may include poly lactic-co-glycolic acid (PLGA) and poly lactic acid (PLA). The biocompatible hydrogel may include at least one of poly ethylene glycol (PEG), poly ethylene glycol diacrylate (PEGDA), and poly ethylene glycol methyl ether acrylate (PEGMEA). The porogen may include at least one of water containing phosphate buffer saline (PBS) or the like, water-soluble hydrogel containing gelatin, and salt or the like solid formulation.

If the skin is pricked on the needle body 12 in the state that the communicating holes 21 of the coating layer 20 are filled with the medicine, the needle body 12 penetrates the outer skin layer and the medicine filled in the communicating holes 21 is delivered to the intradermal layer.

To prove the effects of the microneedle according to an embodiment of the present invention (i.e. an experimental group PC), a comparative group NC was set and tested with respect to a sample medicine, and their efficiencies of delivering the medicine were visualized.

Here, the needle body 12 is made of a stainless material.

Further, the microneedle according to an embodiment of the present invention includes the needle body 12 with the coating layer 20 of which the communicating holes 21 are filled with the sample medicine.

On the other hand, in case of the comparative group, the sample medicine is applied to the surface of the needle body with no coating layer.

At this time, a rhodamin b dye was used as a model of the sample medicine, and gelatin was used as a model of skin.

After the needle body 12 was immersed in the rhodamin b dye to collect the rhodamin b dye on the surface of the needle body 12 and inserted in the gelatin, the delivery of the medicine was examined. In case of the comparative group NC, most rhodamin b dye was moved back on the surface of the gelatin as the needle body 12 is inserted in the gelatin, and thus not properly delivered to the gelatin. On the other hand, the experimental group PC, i.e. the microneedle according to an embodiment of the present invention, a large amount of rhodamin b dye was delivered to the inside of the gelatin after the needle body 12 is inserted in the gelatin.

As shown in FIG. 6, in case where anesthetic medicine, i.e. lidocaine is used as the sample medicine, the experimental group PC, i.e. the microneedle according to an embodiment of the present invention was more improved in efficiency of delivering the medicine by ten or more times than the comparative group NC.

With the foregoing microneedle coated with the porous coating layer, the foregoing manufacturing method thereof and the foregoing microneedle patch with the same, it is possible to secure the channel for delivering the medicine on the surface of the needle body 12 while maintaining the stiffness of the needle body 12 and improve an efficiency of collecting and delivering the medicine based on merits of a material.

Further, it is possible to maximize an efficiency of delivering the medicine when the microneedle is applied to beauty or medical devices or the like.

Further, the medicine is collected in the plurality of communicating holes 21, and it is thus possible to minimize loss of the medicine when the skin is pricked on the needle body 1 and properly deliver the medicine to the intradermal layer even when the needle body 12 is inserted in the skin.

Further, the porosity of the coating layer 20 is controlled, and it is thus possible to continuously supply the medicine even when the needle body 12 is inserted in the skin.

Further, the material for the coating layer 20 may be changed so that the coating layer 20 can be dissolved in a human body to thereby deliver the medicine from the communicating holes 21 to the intradermal layer after the needle body 12 is inserted in the skin.

Further, the efficiency of delivering the medicine is enhanced to have an effect on improving the skin in medical or beauty fields, thereby arousing a market response and increasing utilization in high value-added beauty and medical industries.

Further, the microneedle may be used to not only deliver the medicine but also take a biopsy for extracting a tissue from the outer skin layer or the intradermal layer. At this time, the shape of the communicating holes 21 is controlled to collect a tissue in the communicating holes 21 when the needle body 12 is inserted in and pulled out from the skin.

As described above, the foregoing microneedle coated with the porous coating layer, the foregoing manufacturing method thereof and the foregoing microneedle patch with the same, it is possible to secure the channel for delivering the medicine on the surface of the needle body 12 while maintaining the stiffness of the needle body 12 and improve an efficiency of collecting and delivering the medicine based on merits of a material.

Further, it is possible to maximize an efficiency of delivering the medicine when the microneedle is applied to beauty or medical devices or the like.

Further, the medicine is collected in the plurality of communicating holes 21, and it is thus possible to minimize loss of the medicine when the skin is pricked on the needle body 1 and properly deliver the medicine to the intradermal layer even when the needle body 12 is inserted in the skin.

Further, the porosity of the coating layer 20 is controlled, and it is thus possible to continuously supply the medicine even when the needle body 12 is inserted in the skin.

Further, the material for the coating layer 20 may be changed so that the coating layer 20 can be dissolved in a human body to thereby deliver the medicine from the communicating holes 21 to the intradermal layer after the needle body 12 is inserted in the skin.

Further, the efficiency of delivering the medicine is enhanced to have an effect on improving the skin in medical or beauty fields, thereby arousing a market response and increasing utilization in high value-added beauty and medical industries.

Further, the microneedle may be used to not only deliver the medicine but also take a biopsy for extracting a tissue from the outer skin layer or the intradermal layer. At this time, the shape of the communicating holes 21 is controlled to collect a tissue in the communicating holes 21 when the needle body 12 is inserted in and pulled out from the skin.

Although a few exemplary embodiments of the present invention have been shown and described, it will be appreciated by those skilled in the art that changes may be made in these embodiments without departing from the principles and spirit of the invention, the scope of which is defined in the appended claims and their equivalents.

What is claimed is:

1. A microneedle coated with a porous coating layer, comprising:
a needle body hard enough to penetrate an outer skin layer; and
a coating layer coated to surround the needle body,
wherein the needle body is made of metal,
wherein the coating layer comprises a plurality of communicating holes recessed or penetrated to be filled with medicine,
wherein the plurality of communicating holes communicate with one another to form a moving path of the medicine,
wherein the coating layer is made of a soft material comprising at least one of biocompatible polymer and biocompatible hydrogel; and water-soluble porogen different in state from the soft material,
wherein the communicating holes are recessed or penetrated as the porogent is removed from the coating layer,
wherein the porogen comprises at least one of phosphate buffer saline (PBS), water-soluble hydrogel, and salt,
wherein the needle body is shaped like a horn or a column, a tip of which is formed by its material characteristic.

2. The microneedle coated with the porous coating layer according to claim 1, wherein
the biocompatible polymer comprises at least one of poly lactic-co-glycolic acid (PLGA) and poly lactic acid (PLA), and
the biocompatible hydrogel comprises at least one of poly ethylene glycol (PEG), poly ethylene glycol diacrylate (PEGDA), and poly ethylene glycol methyl ether acrylate (PEGMEA).

3. A method of manufacturing a microneedle of which a needle body is coated with a coating layer,
the method comprising:
an emulsion producing operation of dissolving a soft material as a raw material of the coating layer in a solvent and mixing porogen with the dissolved soft material to produce emulsion;
an emulsion coating operation of coating the emulsion on a surface of the needle body;
an emulsion drying operation of drying the emulsion in the needle body after the emulsion coating operation; and
a porogen removing operation of removing the porogen from the emulsion dried in the needle body, and
the method further comprising a body manufacturing operation of using metal to manufacture the needle body,
wherein the porogen comprises at least one of phosphate buffer saline (PBS), water-soluble hydrogel, and salt.

4. A microneedle patch comprising:
a needle path unit comprising a needle pad shaped like a plate, and a needle body protruding from the needle pad and hard enough to penetrate an outer skin layer; and
a coating layer coated to surround at least the needle body in the needle patch unit;
wherein the needle body is made of metal,
wherein the coating layer comprises a plurality of communicating holes recessed or penetrated to be filled with medicine,
wherein the plurality of communicating holes communicate with one another to form a moving path of the medicine,
wherein the coating layer is made of a soft material comprising at least one of biocompatible polymer and biocompatible hydrogel; and water-soluble porogen different in state from the soft material,
wherein the communicating holes are recessed or penetrated as the porogent is removed from the coating layer,
wherein the porogen comprises at least one of phosphate buffer saline (PBS), water-soluble hydrogel, and salt.
